(19) United States
(12) Patent Application Publication
(10) Pub. No.: US 2010/0316683 A1
(43) Pub. Date: Dec. 16, 2010

(54) HYALURONIC ACID GEL FOR INTRADERMAL INJECTION

(75) Inventors: Estelle Piron, La Bathie (FR); Patrick Bogdanowicz, Balma (FR)

Correspondence Address: BIRCH STEWART KOLASCH & BIRCH, PO BOX 747, FALLS CHURCH, VA, 22040-0747, US

(73) Assignee: PIERRE FABRE DERMO-COSMETIQUE, Boulogne-Billancourt (FR)

(21) Appl. No.: 12/517,862

(22) PCT Filed: Dec. 6, 2007
(86) PCT No.: PCT/EP2007/063384

§ 371 Date: Jul. 20, 2009

(30) Foreign Application Priority Data
Dec. 6, 2006 (FR) 06/10645

(51) Int. Cl. A61K 8/02; A61K 8/73; A61Q 19/08; C08B 37/08

(52) U.S. Cl. 424/401; 514/54; 536/53

(57) ABSTRACT

Implant that is injectable by a sub-cutaneous or intradermal route in the form of a monophasic hydrogel comprising a gel made up of cross-linked hyaluronic acid and one of its physiologically acceptable salts.

The present invention relates to implants based on hyaluronic acid that are injectable sub-cutaneously or intradermally.

Hyaluronic acid (HA) in its acid form or in the form of a salt (hyaluronate) is the major component of the extracellular matrix. It is especially present in the conjunctive tissues referred to as “soft” as opposed to other glycosaminoglycans such as chondroitin sulphuric acid present in the tissues referred to as “hard” such as cartilage. It is found in this way in significant quantities in the skin.

HA is a linear non-sulphated glycosaminoglycan composed of repetitive units of D-glucuronic acid and N-acetyl-D-glucosamine (Tammi R., Agren U. M., Tuhkanen A. L., Tammi M. Hyaluronan metabolism in skin. Progress in Histochemistry & Cytochemistry. 29(2): 1-81, 1994).

In normal skin, HA is synthesized essentially by dermal fibroblasts and epidermal keratinocytes (Tammi R., already cited). By means of residues with a negative charge, HA plays the role of a water pump making it possible to maintain the elasticity of the skin. HA has a major role in the control of the diffusion of nutrients, hormones vitamins and inorganic salts of the conjunctive tissue and in the cleaning of metabolic waste capable of inducing inflammatory reactions. With age, the quantity of HA and its degree of polymerization diminish, resulting in a reduction of the quantity of water retained in the conjunctive tissue. The skin then undergoes an aging process which leads to an increase in fibrosis and a drop in elastic fibre content. In normal human skin, HA exists in the form of a high molecular weight polymer (600,000-1,000,000 Da). The physiological degradation of HA in skin occurs through (i) internalisation by keratinocytes and (ii) intracellular fragmentation into fragments of intermediate size by the hyaluronidases (600,000-300,000 Da). The fragmented HA is released by the keratinocytes, crosses the basal membrane and is released directly into the lymphatic vessels (Tammi R. et al., already cited).

The role of hyaluronic acid (HA) in dermatology is well-known in numerous fields including that of wound healing and hydration. Hyaluronic acid most often acts through its interactions with binding proteins and especially with the transmembrane receptor CD44 (Tool B. P. 2001, Sem. Cell. Devel. Biol. 12: 79-87, Liao Y-H., Stuart A. J., Drug Delivery, 12: 327-342, 2005). Activation of this receptor is expressed through a role in morphogenesis, in cellular multiplication and proliferation, angiogenesis and cellular migration (G. Weindl, M. Schaller, Skin Pharm. Physiol. 2004; 17; 207-213). The data given in the literature suggest that these various actions are a function of the cell environment, of the molecular weight of the hyaluronic acid and of its concentration. For example, the high molecular weights inhibit angiogenesis, while oligosaccharides stimulate it.

Japanese patent application JP 11279042 demonstrates the related action of sulphated hyaluronic acid fragments (HAF) (of molecular mass between 1 and 50 kDa) which would make it possible to maintain skin elasticity and avoid keratinization.

A recent study carried out on HA fragments (of molecular mass between 50 and 750 kDa) relevantly, demonstrates a boosting of the hyaluronic acid synthesis by keratinocytes (patent applications FR 2 865 651, WO 2005 082327). This proliferative activity is induced by HAFs of precise molecular weights, an activity that would not appear with higher or lower molecular weights.

In the field of cosmetology, numerous injectable products based on hyaluronic acid exist. In mesotherapy, solutions of vitamins, antioxidants, inorganic salts or hyaluronic acid are utilised. The vitamins are present to stimulate and maintain cellular metabolism, and thus boost collagen production, the anti-oxidants fight aging and the inorganic salts are indispensable to cellular enzymatic activities. The role of hyaluronic acid is to make it possible to maintain the volume and hydration of the skin and to create a protective screen against degradations by free radicals (H. Trommer, S. Wartewig, Int. Journ. Of Pharm. 254 (2003) 223-234). At sufficient concentrations, hyaluronic acid creates an optimal environment for cellular proliferation and collagen neo-synthesis.

Oxidative stress generating free radicals in the dermis and the epidermis are responsible for skin aging and the appearance of wrinkles, fine lines and tissue sagging.

In addition, it has also been demonstrated that free radicals are also responsible for the depolymerization of hyaluronic acid in situ, which contributes further to the sagging of tissues and their premature aging (Mendoza G., et al, Carbohydrate Research 342, 2007, 96-102).

The extracellular matrix (ECM) is a dynamic structure with a structural and regulatory role for tissues. The ECM is made up of collagen and elastin fibres and also of a fundamental medium (principally water, mineral salts and proteoglycans). This matrix gives the skin its turgidity and its mechanical properties of firmness, elasticity and tonicity.

Collagen macromolecules are fibrous proteins formed from three polypeptide chains linked by covalent and hydrogen bonds. Nineteen types of collagen are known, with half of these in the skin. The majority of dermal collagens belong to the fibrillar collagens I, III and V.

In the young adult, the composition of the dermis is 80% type I collagen and 20% type III collagen; however, this ratio is modified with age as a consequence of aging.

Elastins are proteins organized into fibres within the dermis, they give the skin its properties of elasticity and flexibility. These elastins are rich in amino acids of hydrophobic character.

Degradation of the ECM plays a part during some physiological processes such as wound healing, embryonic development or angiogenesis as well as during pathological situations such as arthritis, atherosis or atherosclerosis, and indeed during phases of tumour progression with formation of metastases (Fisher et al, 1997. N England J. Med., 337, 1419-28; Shapiro, 1998. Current Opinions in Cell Biology, 10, 602-608).

The components of the ECM are principally degraded by endopeptidase enzymes called matrix metalloproteins or MMPs. These MMPs actively contribute to the wound healing process and also contribute to cutaneous relaxation and the appearance of wrinkles which are the first signs of skin aging. The MMP family is comprised of approximately 22 enzymes which are distinguished by specificity with respect to the substrate that they degrade.

MMP-1, or interstitial collagenase, predominantly degrades the triple helix of the type III fibrillar collagens, but also the I, II, VII, VIII and X collagens.

MMP-3 degrades glycoproteins such as fibronectin and laminin, some proteoglycans, elastin, gelatine and collagens IV and V. These two MMPs are expressed by both keratinocytes and fibroblasts.

In the field of filling of wrinkles, chemically cross-linked HA gels are injected intradermally to fill in the depression caused by the wrinkle. The cross-linking makes it possible to increase the persistence of the product inside the dermis. Thus, if the product is correctly injected according to each person's genetic profile, the product allows a filling in 4 to 6 or even 8 months. It is then totally resorbed in the dermis.

Such gels based on HA or cross-linked HA allow the reduction of a wrinkle by a mechanical filling effect in the cutaneous depression resulting from this wrinkle. These products are only endowed with this mechanical effect and do not contribute in any way to any treatment either preventive or curative with respect to skin aging and degradation of the ECM, essential for maintaining the mechanical properties of the skin such as its elasticity and its firmness. Such implants, if they allow eradication of the wrinkles or fine lines, yield an effect that is limited over time and that only partially masks the effects of intrinsic aging of the skin as concerns its maintenance structures represented by the ECM.

To date several products have been used in this same application. Gels or silicone oils are easy to implement, but present the disadvantage of migrating into the tissues located just below the injection points. Chronic inflammation phenomena or allergic reactions have thus resulted. Moreover, silicone is not biodegradable, and ends up in certain organs such as the liver. Different suspensions of polymeric particles have also been proposed, but most have caused rejection reactions, infections, or inflammations. Finally collagen suspensions have been implemented during recent years. However, collagen is resorbed relatively rapidly (between 1 and 3 months), and causes certain allergic reactions due to its origin, being generally of bovine or porcine origin.

The need remains to have injectable implants capable of carrying out wrinkle filling action as well as revitalizing the dermis and epidermis and contributing to the limitation of the processes of cellular senescence accompanying skin aging which do not present the disadvantages mentioned above, or in a much less pronounced manner, the whole being accompanied by a simplicity and increased comfort in implementation.

The present invention therefore relates to a sub-cutaneously or intradermally injectable implant in the form of a monophasic hydrogel, characteristed in that it comprises, by weight, 0.5% to 5%, preferably 0.5% to 4%, more preferably 2% of hyaluronic acid, and wherein:
50% to 95%, preferably 60% to 90%, even more preferably 85% by weight of hyaluronic acid is in cross-linked gel form;
5% to 50%, preferably 10% to 30%, even more preferably 15% by weight of hyaluronic acid is in free form, or in the form of one of the physiologically acceptable salts of HA, of molecular mass between 500 and 2800 kDa, preferably between 750 and 2600 kDa, more preferably between 800 and 2500 kDa, even more preferably between 1000 and 1500 kDa,

in a physiologically acceptable carrier fluid, the ratio between the weight of the gel of cross-linked hyaluronic acid and the weight of free hyaluronic acid being between 1:1 and 1:0.05.

In a particular embodiment of the invention, the implant may comprise approximately 80% of cross-linked hyaluronic acid and approximately 20% of free hyaluronic acid, this principally in the context of an application of the implant according to the invention for the treatment of fine wrinkles. In the case of the treatment of deep wrinkles, the quantity of cross-linked hyaluronic acid will be preferably approximately 85% and the quantity of free hyaluronic acid approximately 15%.

In the context of the present invention, hyaluronic acid or HA is defined as a non-sulphated linear glycosaminoglycan composed of repetitive units of D-glucoronic acid and N-acetyl-D-glucosamine.

Monophasic hydrogel denotes a hydrogel in a single homogeneous phase.

Physiologically acceptable hyaluronic acid salt denotes in particular sodium and potassium salts as well as their mixtures. Advantageously, the salt is sodium salt.

Advantageously, the gel made up of cross-linked hyaluronic acid according to the invention has a viscosity between 200 and 2000 Pa·s., more particularly between 500 and 1800 Pa·s, even more particularly between 1000 and 1800 Pa·s.

In the case of an implant for treatment of deep wrinkles, i.e. comprising 85% of cross-linked hyaluronic acid, the viscosity of the latter is established around 1000 to 1500 Pa·s, particularly around 1200 Pa·s. In the case of an implant principally for mild wrinkles, therefore less loaded with cross-linked hyaluronic acid, i.e. approximately 80% cross-linked hyaluronic acid, the viscosity of such an implant is established around 200 to 500 Pa·s, more particularly around 350 Pa·s.

In this description, the indicated viscosities correspond to values measured for a shear rate of 0.01 s⁻¹.

Advantageously, the cross-linked hyaluronic acid that constitutes the gel according to the invention has a molecular mass between 1000 and 6000 kDa, more advantageously between 1000 and 4000 kDa.

According to an advantageous aspect of the invention, the injectable implant also contains chondroitin sulphate.

Advantageously, the quantity of chondroitin sulphate represents between 0.05% and 5% by weight of the total weight.

Advantageously, the chondroitin sulphate has a molecular mass between 2 and 80 kDa, more advantageously between 20 and 50 kDa.

The implant according to the invention may contain various customary additives. By way of example dyes, colouring pigments, vegetable oils, thickeners, pH modifiers, and osmolarity adjustors may be mentioned.

Free hyaluronic acid, or one of its physiologically acceptable salts, is advantageously distributed homogenously within the cross-linked hyaluronic acid gel.

The carrier fluid is advantageously an apyrogenic sterile isotonic buffer.

The injectable implant according to the invention also advantageously contains at least one other active substance used in dermo-cosmetics.

Advantageously, the dermo-cosmetic active substance is selected from vitamins, anti-oxidants, inorganic salts, antiseptics and chondroitin sulphate.

In a particular embodiment of the invention, the injectable implant contains mannitol which acts as an anti-oxidant at the level of the dermis, and contributes to preserving the HA from depolymerization induced by the free radicals generated within the dermis by oxidative stress. Indeed the association of mannitol with HA has been described as improving protection against damage induced by free radicals (Belda et al, 2005, J. Cataract Refract. Surg., 31: 1213-1218). It is thus an aspect of the present invention to provide an injectable implant as defined above and containing a mannitol as an anti-oxidant.

Another aspect of this invention lies in the use of mannitol within an injectable implant according to the present invention in order to protect the dermis from free radicals and/or to limit depolymerisation of the hyaluronic acid contained therein.

The invention therefore also relates to the use of hyaluronic acid in free form, or in the form of one of its physiologically acceptable salts, of a molecular mass between 500 and 2800 kDa, preferably between 750 and 2600 kDa, more preferably between 800 and 2500 kDa, even more preferably between 1000 and 1500 kDa, in the presence of an anti-oxidant, such as mannitol, for the fabrication of an implant intended to protect the dermis from free radicals and/or to limit depolymerization of the hyaluronic acid of the dermis and/or to prevent skin aging.

The implant according to this invention is injected into the superficial, medium, or deep dermis.

It presents the advantage of being able to cause, at the time of injection, a fibroblastic boost by promoting cellular proliferation and collagen neo-synthesis. Activation of the fibroblasts then generates a modulation of the mechanisms involved in the remodelling of the extracellular matrix, which is expressed by revitalization of the dermis.

The implant according to the present invention presents the double advantage of obtaining a direct and immediate effect of reduction of the wrinkle by mechanical filling of the cutaneous depression and an indirect effect over a longer term of cellular regeneration via a stimulation of collagen synthesis and a regulation of the MMPs.

Cross-linked hyaluronic acid contributes directly to the mechanical filling effect and makes it possible, by its cross-linked nature, to obtain such an effect in a lasting way over periods longer than non-cross-linked HA.

In addition, free HA contained in the implant according to the invention inhibits the over-expression of MMP-1s as well as the over-expression of collagen III. Apart from this regulatory action on the MMP-1s, it contributes to the limitation of the destructuring and destruction of the ECM, an essential structure for the maintenance of the mechanical properties of firmness and elasticity of the skin.

It has been demonstrated, furthermore, that the collagen III/collagen I ratio increases during skin aging (Weber et al, 1984, J. Invest. Dermatol., 82, 156-60). Knowing that within the context of this invention it has been observed that the fraction of free HA used in the implant according to the invention inhibits expression of type III collagen without affecting that of type I collagen, it can reasonably be thought that the implant according to the invention is capable of restoring the collagen III/collagen I ratio to that measured in young tissues.

It is therefore a goal of this invention to use an implant according to this invention for the manufacture of a medicament intended to maintain and or restore the ratio of collagen III/collagen I to that measured in young tissues.

The association of free HA of molecular mass between 500 and 2800 kDa with cross-linked HA gel has the consequence of hydrating and maintaining the volume of the skin in an immediate way. Moreover, it induces a fibroblastic boosting of the dermis, principally due to the presence of free HA, and this fibroblastic boosting is prolonged over time as the in vivo degradation of the cross-linked HA gel progresses.

The implant according to the present invention may be represented as a three-dimensional mesh made up of cross-linked HA containing within it free HA molecules capable of inducing a stimulation of fibroblasts, inhibiting degradation of ECM by inhibition of the MMPs and regulating synthesis of collagen by orienting the latter toward a state corresponding to that observed in young tissue.

These free HA molecules are progressively released from this three-dimensional cross-linked HA matrix as much by passive diffusion as via temporal degradation of the cross-linked HA matrix during the weeks and months following injection. This progressive release makes possible this rejuvenation action of in situ cellular stimulation via these free HA molecules which are preserved from biological degradation within said matrix over the course of several weeks and can thus exert their action over a longer period than if they were injected alone.

Thus the present invention also relates to use hyaluronic acid in its free form, or in the form of one of its physiologically acceptable salts, of a molecular mass between 500 and 2800 kDa, preferably between 750 and 2600 kDa, more preferably between 800 and 2500 kDa, even more preferably between 1000 and 1500 kDa, distributed within a cross-linked hyaluronic acid gel, for the fabrication of a sub-cutaneous implant for the filling of wrinkles and the epidermal cellular boosting and/or maintaining the mechanical properties of firmness and elasticity of the skin.

Therefore the implant according to the invention combines the mechanical effect of the cross-linked gel, which swells and reshapes the wrinkle, with the biological action of free HA.

The invention also relates to a kit in the form of a syringe containing an injectable implant such as previously described.

The invention also relates to the implant as previously described as a medicament.

The invention also relates to the use of an injectable implant as previously described for the filling of wrinkles, fine lines, cutaneous depressions and/or scars comprising the sub-cutaneous injection of such an implant.

The injectable implant according to the invention may be used for the preparation of a medicament intended to stimulate epidermal and dermal metabolism and/or to boost epidermal cellular activity.

The injectable implant according to the invention may be used for the preparation of a medicament intended to stimulate the anti-oxidant activity of the dermis and/or prevent cutaneous aging.

The invention also relates to a cosmetic method for filling wrinkles and/or fine lines, comprising the injection of at least one injectable implant according to the invention.

The invention also relates to a method for the preparation of an injectable implant as previously described. It may be prepared by any technique known to those skilled in the art, for example by a diepoxy, in particular butanediol diglycidyl ether (BDDE) or 1,2,7,8-diepoxy-octane. With cross-linking in a basic medium, the diepoxy concentration may vary for example between 5 and 15% with respect to hyaluronic acid for cross-linking in a water bath at 45-55° C. for 1.5 to 6 hr. The cross-linked gel is then purified by classic techniques of those skilled in the art: various deionized water baths, alcoholic precipitation, dialysis, etc. to eliminate traces of residual cross-linking agent. To this purified gel, HA of appropriate molecular weight, previously hydrated in an appropriate buffer, will be added. The finished product will finally be degassed, put in a syringe or any other appropriate container and sterilized by autoclaving.

An advantageous procedure according to the invention comprises the following steps:

1) preparation of a cross-linked gel according to the following steps:
addition of hyaluronic acid to a basic fluid,
swelling, homogenization under slow agitation and hot cross-linking,
neutralization and swelling of the cross-linked gel in a buffered solution at a pH of approximately 7 with the addition of an iso-osmolarizing agent,
elimination of the cross-linking agent,

2) preparation of a free hyaluronic acid gel by:
addition of hyaluronic acid to a buffered solution with pH of approximately 7, iso-osmolar:
swelling,

3) mixture of the cross-linked gel obtained in step 1) with the free hyaluronic acid gel obtained in step 2),

4) optional degassing and possible packaging in bottles or syringes and then sterilization.

Advantageously, the pH of the gel after sterilization is approximately 7, and the osmolarity is around 250 to 350 mOsm, preferably between 300 and 320 mOsm.

The invention will now be illustrated, in a non-restrictive way, by the following examples.

### EXAMPLE 1

Characterization of HA activity of various molecular masses on:
healthy fibroblasts;
senescent fibroblasts (by oxidative stress with H₂O₂).

MMP-1 (collagenase 1) is an interstitial collagenase which degrades the triple helix of fibrillar collagens such as collagens I and III. In the skin, it is expressed and secreted by fibroblasts and keratinocytes. MMP-1 is involved in aging. Indeed, its over-production during aging could be involved in the loss of firmness and elasticity, and the appearance of wrinkles. When senescence is induced with H₂O₂, a large increase in MMP-1 is observed in fibroblasts. The principle active substances capable of reducing this overproduction could therefore have “anti-aging” properties.

Fibroblasts were obtained from skin derived from operating room remainders from young subjects. The samples were washed in PBS and ethanol. Small pieces of skin were cut up and distributed in culture dishes and immersed in a medium favourable to the proliferation of fibroblasts (DMEM+10% SVF). Primocin was added to this medium. The latter is an antibiotic, antifungal and anti-micoplasma. The culture dishes are put in the incubator.

To induce senescence, fibroblasts were seeded in culture dishes. After 24 hours, the active substance to be tested is added at the appropriate concentration, to DMEM. After 24 hours of incubation, the cells are subjected to an oxidizing stress. They were incubated for 2 hours at 37° C., in a 75 μM H₂O₂ PBS solution. Acute stress that induces senescence of the cells is involved. The fibroblasts were then put back into complete DMEM with 10% of SVF. The fibroblasts return to senescence 72 hours after the end of the stress.

1.1 Incubation Protocol.

For senescent fibroblasts: the HAs are added to a final concentration of 1 μg/ml, they are left during the H₂O₂ stress, that is, 24 hr. of incubation.

For healthy fibroblasts: the HAs are also incubated for 24 hour at 1 μg/ml.

1.2 Extraction of Total RNA by Means of the Rneasy Extraction Kit (Qiagen) at the End of the Experiment (Day 5, i.e. 72 hr. post-stress).

1.3 Assay of Total RNA

The qualitative and quantitative assay of total RNAs is carried out by means of the RNA 6000 Nano LabChip assay kit and the Bioanalyzer 2100 apparatus (Agilent). It is based on the principal of electrophoretic migration of the samples in a nanochip. The ratio of 28S/18S ribosomal RNAs is calculated; it is informative in evaluating the integrity of the RNAs. Moreover, the purity of the RNAs with respect to the genomic DNA is estimated.

1.4 Analysis of the Rate of Transcription by Real Time PCR.

The real time PCR makes it possible to quantify the level of transcriptional expression of a gene of interest by amplification of the cDNAs, obtained by reverse transcription, of the mRNAs of the gene present in the cell lysate. The reaction mixture is made up of cDNA and of the mixture of the couple of primers of interest with a solution of iQ SvbrGreen Supermix (Biorad) containing, among others, Taq Polymerase and an intercalating agent of the small groove of the DNA double helices: the Sybr Green (fluorescent intercalating agent). The reaction is performed in the Icycler IQ thermocycler (Biorad); a succession of denaturation/hybridization/elongation cycles is involved.

1.4.1. Reverse Transcription: the Total RNAs are “retro-transcribed” into cDNA by Means of the Reverse Transcription System (Promega) and the Gene Amp PCR System 2400 (Perkin Elmer).

1.4.2. Measurement of the rate of transcription of a gene of interest: the level of expression of the gene of interest is normalized by the level of expression of the reference genes, which varies little with the senescence. The level of expression of the gene of interest is calculated according to formula: 2 (CTmin-CT) where CT signifies “cycle threshold.”

It is normalized, with respect to the expression of the three reference genes selected, according to calculation: 2 (CTmin-CT)/normalization factor.

1.5. Results on the Expression of MMP-1.

The fibroblasts made senescent by incubation with H₂O₂ expressed 2.14 times more MMP-1 than normal fibroblasts (therefore senescence has been properly induced). In the presence of Vitamin E (positive control), the overexpression of MMP-1 by the senescent fibroblasts was reduced by 40%.

The overexpression of MMP-1 was reduced by 40% in the presence of HA 450 kDa (10 μg./ml), by 75% in the presence of HA 800 kDa (10 μg./ml), by 71% in the presence of HA 1500 kDa (10 μg/ml), and by 83% in the presence of HA 2600 kDa (10 μg./ml).

1.6. Results on the Expression of Type I and Type III Collagen.

The fibroblasts made senescent by incubation with H₂O₂, a variable inhibition of the transcription of type I collagen was measured; such an inhibition seemed however dependent on the donors. On one of the donors for which the transcription of the type I was inhibited by 24% by the H₂O₂ stress, the various HAs of various sizes wouldn't restore the synthesis of collagen.

In fibroblasts made senescent, an increase of +163% to +304% of the transcription of type III collagen was measured. On the donor for which the transcription of the type III collagen is stimulated by +304% by the H₂O₂ stress, the free HAs of various sizes inhibit the collagen synthesis. The HA of 450 kDa inhibits by approximately 25%, whereas the HAs of mass greater than 800 kDa inhibit the transcription by approximately 100%, bringing it back to levels close to that of young fibroblasts.

1.7. Conclusion.

The conclusion of this experiment is that, it appears that the HAs have an anti-MMP-1 activity (hence anti-oxidant therefore having potentially an anti-aging activity) in this model of induced senescence. The HAs of higher molecular mass (800, 1500 and 2600 kDa) seem more effective than the HA of 450 kDa.

Under present experimental conditions, the HAs of molecular mass 450 KDa are active on the MMP-1. The HAs of molecular mass greater than 800 kDa are more active since they inhibit the overexpression of −75 to −83%.

On the other hand, these results show that the various free HAs do not act on the synthesis of type I collagen.

Finally, the free HAs inhibit the overexpression of type III collagen, and this is particularly remarkable for the HAs of molecular mass of 800 kDa and above, which inhibit the overexpression by −118 to −93%.

Thus, in addition to their regulatory action on the MMP-1s, the free HAs contained in the implant according to this invention contribute to limiting the destruction and the destructuring of the MEC. Furthermore, knowing that it was demonstrated that the ratio of collagen III/collagen I increased during aging, the HAs contained in the implant according to the invention make it possible to restore the ratio measured in young tissues.

### EXAMPLE 2

### Preparation of an Implant According to the invention

2.1. Preparation of Cross-Linked HA.

5 g of sodium hyaluronate of molecular weight of 1.6 MDa have been added to 1% sodium hydroxide (35.6 g). The mixture is left to homogenize discontinuously for 1 hr. and 30 minutes. Then, 315 mg of BDDE were added to the HA/NaOH mixture which has been homogenised, closed and then placed in a water-bath at 50° C. for 2 hr. The mixture was neutralised by the addition of 5 g of 1N HCl.

The gel thus obtained was added at the desired concentration by addition of EDI and salts guaranteeing iso-osmolarity, as well as a stable neutral pH to give a gel at 20 mg/g in HA.

2.2. Preparation of the Implant.

To this purified gel is added HA of 1.2 MDa, previously hydrated in phosphate buffer. Then 60 g of HA gel 1.2 MDa, of concentration 20 mg/g are added to the 226 g of cross-linked gel obtained. The 2 gels are homogenized in a standard blade mixer for 1 to 2 hours.

The finished product may finally be degassed, packed in syringes and sterilised by steam autoclaving by following a cycle for example of 125° C. for 7 min, 127° C. for 4 min. or 130° C. for 3 min.

After sterilization, the pH of the product is 7.1, osmolarity of 320 mOsm, and the rheological characterisations give an elastic modulus G′ of 45 Pa·s at a frequency of 1 Hz. The final concentration of the gel is assayed to 19.8 mg/g (assay with carbazole, according to the method of the European Pharmacopoeia), for an expected concentration of 20 mg/g.

1. Implant that is injectable sub-cutaneously or intradermally in the form of a monophasic hydrogel, characterized in that it comprises, by weight, 0.5% to 5%, preferably 0.5% to 4%, more preferably 2% hyaluronic acid, and wherein:
50% to 95%, more preferably 60% to 90%, even more preferably 85% by weight of hyaluronic acid is in the form of a cross-linked gel,
5% to 50%, preferably 10% to 30% even more preferably 15% by weight of the hyaluronic acid is in the free form or in the form of one of its physiologically acceptable salts, of a molecular mass between 500 and 2800 kDa, preferably between 750 and 2600 kDa, more preferably between 800 and 2500 kDa, even more preferably between 1000 and 1500 kDa,
in a physiologically acceptable carrier fluid, the ratio between the weight of the cross-linked hyaluronic acid gel and the weight of the free hyaluronic acid being between 1:1 and 1:0.05.

2. Injectable implant according to claim 1, characterized in that the gel made up of cross-linked hyaluronic acid has a viscosity between 200 and 2000 Pa·s, preferably between 1000 and 1800 Pa·s.

3. Injectable implant according to claim 1, characterized in that the hyaluronic acid making up the cross-linked gel has a molecular mass between 1000 and 6000 kDa, preferably between 1000 and 4000 kDa.

4. Injectable implant according to claim 1, characterized in that it further contains chondroitin sulfate having a molecular mass between 2 and 80 kDa, preferably between 20 and 50 kDa.

5. Injectable implant according to claim 1, characterized in that the free hyaluronic acid, or one its physiologically acceptable salts, is distributed homogeneously inside the cross-linked hyaluronic acid gel.

6. Injectable implant according to claim 1, characterized in that the carrier fluid is an apyrogenic sterile isotonic buffer.

7. Injectable implant according to claim 1, characterized in that it contains moreover at least one other active substance used in dermo-cosmetics.

8. Injectable implant according to claim 8, characterized in that the dermo-cosmetic active substance is selected from vitamins, anti-oxidants, inorganic salts, antiseptics and chondroitin sulfate.

9. Injectable implant according to claim 8, characterized in that the anti-oxidant is mannitol.

10. Injectable implant according to claim 1 as medicament.

11. Kit presented in syringe form and containing an injectable implant according to claim 1.

12. Use of hyaluronic acid in free form, or in the form of one of its physiologically acceptable salts, of molecular mass between 500 and 2800 kDa, preferably between 750 and 2600 kDa, more preferably between 800 and 2500 kDa, even more preferably between 1000 and 1500 kDa, in the presence of an anti-oxidant, such as mannitol, for the manufacture of an implant intended to protect the dermis from free radicals and/or to limit the depolymerization of the hyaluronic acid of the dermis.

13. Use of an injectable implant according to claim 1, or of the kit, for the filling of wrinkles, fine lines, cutaneous depressions and/or scars comprising sub-cutaneous injection of such an implant.

14. Use of an injectable implant according to claim 1, for the preparation of a medicament for stimulating the anti-oxidant activity of the dermis and/or to prevent skin aging.

15. Use of an implant according to claim 1, for the manufacture of a medicament for maintaining and/or restoring the collagen III/collagen I ratio to that measured in young tissues.

16. Use of hyaluronic acid in free form, or in the form of one of its physiologically acceptable salts, of a molecular mass between 500 and 2800 kDa, preferably between 750 and 2600 kDa, more preferably between 800 and 2500 kDa, even more preferably between 1000 and 1500 kDa, distributed within a gel of cross-linked hyaluronic acid, for the manufacture of a sub-cutaneous implant intended for the filling of wrinkles and for the boosting of the epidermal cellular activity and/or for the maintenance of the mechanical properties of firmness and elasticity of the skin and/or the stimulation of the epidermal and dermal metabolism and/or to stimulate anti-oxidant activity of the dermis and/or prevent skin aging.

17. Cosmetic method for the filling of wrinkles and/or fine lines, comprising injection of at least one injectable implant according to claim 1.

18. Preparation method of an injectable implant according to claim 1, characterized in that it comprises the following steps:
1) preparation of a cross-linked gel according to the following steps:
addition of hyaluronic acid to a basic fluid,
swelling, homogenization under slow agitation and hot cross-linking,
neutralization and swelling of the cross-linked gel in a buffered solution at a pH of approximately 7 with addition of iso-osmolarizing agent,
elimination of the cross-linking agent,
2) preparation of a free hyaluronic acid gel by:
addition of hyaluronic acid to a buffered solution with a pH of around 7, iso-osmolar;
swelling,
3) mixture of the cross-linked gel in step 1) with the free hyaluronic acid gel obtained in step 2).
4) optional degassing and optional packaging in bottles or syringes and then sterilization.
